(19) United States
(12) Patent Application Publication
(10) Pub. No.: US 2025/0195452 A1
(43) Pub. Date: Jun. 19, 2025

(54) ER-BETA ESTROGENIC COMPOUNDS AND METHODS OF USE

(71) Applicant: laterion, Inc., Oakland, CA (US)

(72) Inventors: Isaac Cohen, Oakland, CA (US); Dale C. Leitman, Oakland, CA (US)

(21) Appl. No.: 19/038,620

(22) Filed: Jan. 27, 2025

Related U.S. Application Data
(63) Continuation of application No. PCT/US23/28880, filed on Jul. 27, 2023.
(60) Provisional application No. 63/393,640, filed on Jul. 29, 2022.

(51) Int. Cl. A61K 31/12

(52) U.S. Cl. CPC A61K 31/12

(57) ABSTRACT

Estrogenic compounds, combinations, pharmaceutical compositions and methods are presented. The estrogenic compounds comprise ERβ-selective estrogenic compounds and combinations thereof. The novel compositions comprise one or more of the ERβ-selective estrogenic compounds, and may optionally comprise one or more excipients. The one or more excipients may be pharmaceutically acceptable. The one or more excipients may comprise at least one compound that does not occur in nature with estrogenic compounds. The method comprises administering a pharmaceutical composition comprising an estrogenic compound described herein to a patient in need of treatment with an estrogenic compound or combination, or a pharmaceutical composition comprising the estrogenic compound or combination.

### CROSS-REFERENCE

This application claims the benefit of U.S. Provisional Application No. 63/393,640 filed Jul. 29, 2022, the contents of which are incorporated herein by reference in their entirety.

### FIELD

The present invention is directed to estrogenic compounds, compositions, and methods, including compositions for use in the prevention, treatment, or reduction in severity and/or frequency symptoms of menopause.

### BACKGROUND

Estrogen is a steroid hormone involved in control of multiple developmental and physiological function in both males and females. Estrogen is not just an estrus-inducing sex hormone. Estrogens are pivotal for survival and health in both genders, though their quantity, adaptive response, tissue-specific distribution, and receptor affinity vary with different phases of life. Estrogen is indispensable to glucose homeostasis, immune robustness, bone health, cardiovascular health, fertility, and neural functions.

Estrogens (estradiol (E2) and related steroidal hormones) are biosynthesized mainly in the gonads but also in the adrenal cortex and adipose tissue. Estrogen regulation is mediated by nuclear estrogen receptors (ERs) and the membrane estrogen receptor (GPER1), which are encoded by different genes on different chromosomes, suggesting differential functions. However, estrogen is at the center of almost all human pathologies as well—e.g., infectious, autoimmune, metabolic and degenerative conditions. Both hypo- and hyper-estrogen levels have been linked to chronic and acute diseases. While normal aging results in a significantly lower level of estrogen, leading to tissue degeneration (bone, muscle, neural etc.), and metabolite imbalance (glucose, lipid etc.), the increase in inflammatory agents in day-to-day life enhances the level of estrogen (or estrogen mimic), which is of critical pathophysiological importance. The resultant excess estrogen induces fluctuations in estrogen receptors alpha and beta (ERα and ERβ), harming tissues, leading to autoimmune diseases, and neoplasms.

Estrogen is involved in growth, development, and tissue differentiation from the embryonic phase to death. Puberty gives rise to rapid physiological changes like a growth spurt of bone and muscle mass, and maturation of the gonads and the brain. Estrogen is a crucial signaling contributor to all these processes and especially to sexual differentiation. Hormone fluctuations and disruptions lead to multiple pathologies at this age, not all of which are directly associated with just sexual development and function. There is a world-wide increase in pre-mature puberty which results in increased female cancer risks and reduces girl-specific quality of life.

During menarche estrogen levels fluctuate significantly throughout the menstrual cycle. Early menarche and extended menarche, even at normal levels of cycle fluctuations, result in significant increase in risk of aging diseases such as breast cancer and type 2 diabetes. A majority of women (up to 91%) experience painful menstruation and 14% experience heavy bleeding. There is a world-wide increase in female infertility, which is not just due to increase in age of first pregnancy. Up to 21% of couples suffer infertility, with up to 80% due to female disorders. Despite improved fertility technologies such as in-vitro fertilization), the success rate of these technologies is very low (approximately 9% for IVF). Up to 46% of women experience spontaneous abortion (miscarriage). There is increasing trend of gestational diabetes, preterm labor, preeclampsia and gestational anemia, all associated with estrogen function.

In women of reproductive age, estrogen is mainly synthesized in the granulosa cells of ovarian follicles. As women enter menopause, the follicles in the ovaries are depleted due to atresia during each menstrual cycle, and the amount of estrogen produced by the ovaries declines. When estrogen levels begin to drop and fluctuate, short-term symptoms such as hot flashes, night sweats, and mood changes frequently arise. The ovaries will eventually stop producing estrogen, and the duration of estrogen deficiency will increase, accelerating the risk of chronic diseases such as osteoporosis, cardiovascular disease, obesity, type 2 diabetes, and urogenital atrophy.

Approximately 70% of all autoimmune diseases are diagnosed in women including rheumatoid arthritis, lupus, Sjogren's syndrome, scleroderma, Hashimoto's thyroiditis, multiple sclerosis among others. As women age there is increased incidence of breast cancer and ovarian cancer. More so, approximately 70% of cases of Alzheimer's disease and early dementia are diagnosed in women.

Estrogen pharmaceutical intervention, e.g. menopausal hormone therapy (MHT), is the most common intervention in all female-related disorders. Natural estrogen and synthetic derivatives are used in these prescriptions. The mechanism of action of these estrogens are as universal agonists, resulting in activation and repression of genes. There are several synthetic steroidal and non-steroidal estrogen receptor antagonists used mostly for the treatment of breast cancer. Despite the pharmacological intention to achieve estrogen receptor antagonism, resulting in gene regulation and pathophysiological outcome, which is unlike the natural hormone, most steroidal and non-steroidal drugs result in a mixed agonist/antagonist outcome, whereby they result in opposite outcome to estrogen in some tissues, while resulting in similar outcome in other tissues. These are the selective estrogen receptor modulators (SERMs), now used for the treatment of breast cancer, osteoporosis and, when combined with estrogen, for menopausal symptoms.

Historically, to oppose the increase in uterine cancer risk associated with menopausal hormone therapy, progestogens are co-prescribed with estrogens. Progestogens exert their pharmacological action via the progesterone receptor.

In recent decades, since the discovery of the ERβ in 1995, attempts have been made to develop receptor subtype agonist and antagonist drugs. None of these drugs are approved by regulators.

Menopausal hormone therapy (MHT) is recommended for only five years to treat menopausal and perimenopausal vasomotor symptoms and vulvovaginal atrophy, as longer term administration of MHT has been shown to increase the risk of estrogen-related adverse outcomes, such as breast cancer, stroke, venous thrombosis (VTE), and Alzheimer's disease. Nonetheless, treatment with MHT, in addition to treating vasomotor symptoms of menopause, also reduces the risk of osteoporosis and type 2 diabetes mellitus (T2DM), cardiovascular disease, obesity, and urogenital atrophy. Thus, there is a need for therapeutic options having one or more advantages of MHT with decreased risk of adverse outcomes, such as increased cancer risk associated with traditional MHT (E2, alone or on combination with a progestogen, or a Selective Estrogen Receptor Modulator (SERM)).

There is a need for additional and novel therapeutic compounds, compositions, and methods for the treatment of symptoms of menopause and perimenopause. The various embodiments disclosed herein address these needs and provide related advantages as well.

### BRIEF SUMMARY OF THE INVENTION

The compounds described herein, alone or in combination with each other or with other estrogens, can be used for multiple indications as they result in modification of tissues expressing ERβ. Activation of ERα by estradiol results in dose dependent increase of proliferation of breast and uterine cancer cells as well as a dose dependent increase in uterine hyperplasia and weight. Unlike estradiol activity on ERα, estradiol inhibits the growth breast and uterine cancer cells expressing ERβ in a dose dependent manner and in no uterine hyperplasia or increase of uterine weight, suggesting that use of ERβ-specific ligands will augment estrogen activity and reduce the risk of breast and uterine cancer.

Thus, provided herein are compounds and pharmaceutically acceptable salt thereofs, wherein the compounds are ERβ-selective estrogenic compounds. In some embodiments, each ERβ-selective estrogenic compound is a member of the group consisting of: Liquiritigenin; Apigenin; Luteolin; Galangin; Naringenin; Calycosin; 6-methoxyluteolin; Nyasol; Wogonin; Broussonin A; 7,4′-Dihydroxyflavone; 6,4′-Dhydroxyflavone; 5,4′-Dihydroxyflavone; 3,4′-Dihydroxyflavone; 7,2′-Dihydroxyflavone; 7,3′-Dihydroxyflavone; Analog 13; Genistein; or Equol, or a pharmaceutically acceptable salt of any of the foregoing. In some embodiments, the ERβ-selective compound is not an agonist, an antagonist or a mixed agonist/antagonist of estrogen receptor alpha. Also provided are combinations comprising two or more ERβ-selective compounds, or pharmaceutically acceptable salts thereof, of the same group. Also provided are pharmaceutical compositions comprising (1) one or more pharmaceutically acceptable ingredients other than an ERβ selective compound and (2) one or more ERβ-selective compounds. In some embodiments, at least one pharmaceutically acceptable ingredient other than an ERβ-selective compound is estradiol or other ERα-modulating compound. In some embodiments, at least one pharmaceutically acceptable ingredient other than an ERβ-selective compound does not occur naturally with the ERβ-selective estrogenic compound in nature. In some embodiments, the pharmaceutical composition comprises a member of the group consisting of estradiol (E2), one or more estrogen receptor agonists, one or more estrogen receptor antagonists, one or more mixed estrogen receptor agonist/antagonists, one or more selective estrogen receptor modulators (SERMs), one or more progestogens, one or more glucocorticoids, and one or more androgens. In some embodiments, the compound, combination or pharmaceutical composition is intended for use in treatment of a condition or disease state in a patient in need of treatment with a nuclear receptor reprogramming compound. In some embodiments, the condition or disease state is a development condition, a menstruation condition, a fertility condition, a gynecological disease, an autoimmune disorder, a menopause disorder, an aging disorder, or cancer. In some embodiments, the condition or disease state is Turner Syndrome, Kallman Syndrome, congenital primary amenorrhea, a childhood neuropsychiatric disorder, dysmenorrhea, amenorrhea, menorrhagia, estrogen-induced deep vein thrombosis, pulmonary embolism, conception, fetal implantation, spontaneous abortion, preterm labor, endometriosis, polycystic ovarian syndrome, rheumatoid arthritis, scleroderma, Sjogren's syndrome, Hashimoto's thyroiditis, multiple sclerosis, irritable bowel syndrome, ulcerative colitis, Crone's disease, vasomotor symptoms of menopause and perimenopause, insomnia, nighttime wakening, mood swings, vulvovaginal atrophy, vaginal dryness, dyspareunia, menopausal weight gain and obesity, osteoporosis, type-2 diabetes, estrogen-induced deep vein thrombosis and pulmonary embolism, Alzheimer's disease, early dementia, breast cancer, uterine cancer, ovarian cancer, prostate cancer, and non-small cell lung cancer.

Some embodiments described herein comprise use of a compound, combination or a pharmaceutical composition described in the immediately-preceding paragraph for manufacture of a medicament for use in treatment of a condition or disease in a patient in need of treatment with a nuclear receptor reprogramming compound. In some embodiments, the condition or disease state is a development condition, a menstruation condition, a fertility condition, a gynecological disease, an autoimmune disorder, a menopause disorder, an aging disorder, or cancer. In some embodiments, the condition or disease state is Turner Syndrome, Kallman Syndrome, congenital primary amenorrhea, a childhood neuropsychiatric disorder, dysmenorrhea, amenorrhea, menorrhagia, estrogen-induced deep vein thrombosis, pulmonary embolism, conception, fetal implantation, spontaneous abortion, preterm labor, endometriosis, polycystic ovarian syndrome, rheumatoid arthritis, scleroderma, Sjogren's syndrome, Hashimoto's thyroiditis, multiple sclerosis, irritable bowel syndrome, ulcerative colitis, Crone's disease, vasomotor symptoms of menopause and perimenopause, insomnia, nighttime wakening, mood swings, vulvovaginal atrophy, vaginal dryness, dyspareunia, menopausal weight gain and obesity, osteoporosis, type-2 diabetes, estrogen-induced deep vein thrombosis and pulmonary embolism, Alzheimer's disease, early dementia, breast cancer, uterine cancer, ovarian cancer, prostate cancer, and non-small cell lung cancer.

Some embodiments described herein provide a method of treating a patient in need of such treatment, comprising administering to the patient an effective amount of a compound, or a pharmaceutically acceptable salt thereof, a combination, or a pharmaceutical composition described in either of the two immediately-preceding paragraphs. In some embodiments, the condition or disease state is a development condition, a menstruation condition, a fertility condition, a gynecological disease, an autoimmune disorder, a menopause disorder, an aging disorder, or cancer. In some embodiments, the condition or disease state is Turner Syndrome, Kallman Syndrome, congenital primary amenorrhea, a childhood neuropsychiatric disorder, dysmenorrhea, amenorrhea, menorrhagia, estrogen-induced deep vein thrombosis, pulmonary embolism, conception, fetal implantation, spontaneous abortion, preterm labor, endometriosis, polycystic ovarian syndrome, rheumatoid arthritis, scleroderma, Sjogren's syndrome, Hashimoto's thyroiditis, multiple sclerosis, irritable bowel syndrome, ulcerative colitis, Crone's disease, vasomotor symptoms of menopause and perimenopause, insomnia, nighttime wakening, mood swings, vulvovaginal atrophy, vaginal dryness, dyspareunia, menopausal weight gain and obesity, osteoporosis, type-2 diabetes, estrogen-induced deep vein thrombosis and pulmonary embolism, Alzheimer's disease, early dementia, breast cancer, uterine cancer, ovarian cancer, prostate cancer, and non-small cell lung cancer.

Other uses and advantages of the various embodiments described herein will be apparent to those skilled in the art upon review of the following disclosure.

### BRIEF DESCRIPTION OF THE DRAWINGS

The features and advantages of the present invention will become apparent from a consideration of the following detailed description presented in connection with the accompanying drawings in which:

FIG. 1 shows luciferase activity of several compounds of the invention in U2OS cells transfected with NKG2E TK-Luc and ERβ.

FIG. 2 shows dose response for several compounds of the invention U2OS cells transfected with NKG2E TK-Luc and ERβ.

FIGS. 3 and 4 show the structures of several compounds of the instant invention. The basic flavone structure with numbering of ring positions is in the top left corner of FIGS. 3 and 4.

FIG. 5 shows luciferase activity dose response for ligands on the activation of the NKG2E-TK-Luc in U2OS-ERβ Cells.

FIGS. 6 and 7 show compounds of the invention. Ligands enclosed in boxes (Liquiritigenin, Apigenin, Nyasol, Naringenin, 7,4′-Dihydroxyflavone, 6,4′-Dihydroxyflavone, 5,4′-Dihydroxyflavone, Analog 13, and Genistein) activate ERβ at 200 nM. The basic flavone structure with numbering of ring positions is in the top left corner of FIGS. 6 and 7.

FIG. 8 shows dose responses for ligands on the Activation of the NKG2E ERE in U2OS-ERα Cells.

FIGS. 9 and 10 show compounds of the invention. Ligands that activate ERα at 1 μM (Apigenin, Nyasol, Broussonin A, 6,4′-Dihydroxyflavone, Genistein, and Equol) are enclosed in double boxes and those that activate ERα at 5 μM (Liquiritigenin, 7,4′-Dihydroxyflavone and Analog 13) are enclosed in single boxes. The basic flavone structure with numbering of ring positions is in the top left corner of FIGS. 9 and 10.

FIG. 11: Shows keratin 19 mRNA expression in U2OS-ERβ Cells by qRT-PCR.

FIG. 12A Shows luciferase activity for U2OS-ERβ cells transfected with GRE-TK-Luc and GR in the presence of doxycycline.

FIG. 12B Shows luciferase activity for U2OS WT cells transfected with GRE-TK-Luc and ERβ in the presence of GR.

FIG. 13A shows GILZ expression in the presence of Liquiritigenin and/or doxycycline in U2OS-ERβ cells expressing GR.

FIG. 13B shows Ladinin expression in the presence of Liquiritigenin and/or doxycycline in U2OS-ERβ cells expressing GR.

FIG. 14 shows luciferase activity in U2OS-ERβ cells expressing TAT3-TK-Luc and hPRβ in the presence of ERβ ligands and combinations.

FIG. 15 shows the potency of ERβ ligands in MF101.

FIGS. 16A and 16B show histology results for ERβ ligands IATERB5, IATERB6 (FIG. 16A) and IATERB7 (FIG. 16B) in comparison to E2. Unlike estradiol, three of the ERβ compounds do not increase uterine breast cancer tumor formation.

FIG. 17 shows dose response luciferase activities for ERβ ligands, alone and in combination with hydrocortisone (HC) in U2OS-ERβ cells transfected with GRE-TK-Luc, GR, and doxycycline.

### DETAILED DESCRIPTION OF THE INVENTION

Liquiritigenin; Apigenin; Luteolin; Galangin; Naringenin; Calycosin; 6-methoxyluteolin; Nyasol; Wogonin; Broussonin A; 7,4′-Dihydroxyflavone; 6,4′-Dhydroxyflavone; 5,4′-Dihydroxyflavone; 3,4′-Dihydroxyflavone; 7,2′-Dihydroxyflavone; 7,3′-Dihydroxyflavone; Genistein; and Equol may be obtained from commercial sources, e.g., Sigma-Aldrich. Analog 13 may be obtained by the synthetic method set forth in the Examples, hereinafter.

Compounds of the invention are summarized in Table 1:

TABLE 1
ERβ-Selective Compounds

| Iaterion Designator | Common Name |
|---|---|
| IATERB1 | Galangin |
| IATERB2 | 6-methoxyluteolin |
| IATERB3 | Wogonin |
| IATERB4 | Broussonin A |
| IATERB5 | Nyasol |
| IATERB6 | Calycosin |
| IATERB7 | Liquiritigenin |
| IATERB8 | Naringenin |
| IATERB9 | Apigenin |
| IATERB10 | Luteolin |
| IATERB11 | 5,4′ Dihydroxyflavone |
| IATERB12 | 6,4′ Dihydroxyflavone |
| IATERB13 | 3,4′ Dihydroxyflavone |
| IATERB14 | 7,4′ Dihydroxyflavone |
| IATERB15 | 7,3′ Dihydroxyflavone |
| IATERB16 | 7,2′ Dihydroxyflavone |
| IATERB17 | Analog 13 (A13) |

### Pharmaceutical Compositions

Pharmaceutical compositions described herein comprise one or more compounds, e.g. two or more compounds, three or more compounds, four or more compounds, five or more compounds, six or more compounds, or seven or more compounds selected from the group consisting of: Liquiritigenin; Apigenin; Luteolin; Galangin; Naringenin; Calycosin; 6-methoxyluteolin; Nyasol; Wogonin; Broussonin A; 7,4′-Dihydroxyflavone; 6,4′-Dhydroxyflavone; 5,4′-Dihydroxyflavone; 3,4′-Dihydroxyflavone; 7,2′-Dihydroxyflavone; 7,3′-Dihydroxyflavone; Analog 13; Genistein; or Equol, or a pharmaceutically acceptable salt of any of the foregoing. At least one of the pharmaceutically acceptable ingredients may include one or more ingredients that do not naturally occur with the ERβ-selective compounds disclosed herein, a salt or a solvate thereof. Pharmaceutically acceptable ingredients that do not naturally occur with the novel compounds disclosed herein, a salt or a solvate thereof, in nature may include sterile, isotonic, or pyrogen free excipients.

A “pharmaceutically acceptable” ingredient is an ingredient that is compatible with the estrogenic compounds as described herein and with other ingredients of the composition and is suitable for administration to a patient. Additional ingredients may include carriers, diluents, absorption enhancers, stabilizers, preservatives, or other active or inactive ingredients. At least one of the additional ingredients may be an ingredient that does not occur naturally with an ERβ-compound or combination of ERβ-compounds as described herein in nature. At least one of the additional ingredients may be an ingredient other than water. In some embodiments, the pharmaceutical composition may be sterile, pyrogen free, and/or isotonic. In some embodiments, the pharmaceutical composition is sterile or pyrogen free. In some embodiments, the pharmaceutical composition is sterile and pyrogen free. In some preferred embodiments, the pharmaceutical composition is sterile, pyrogen free and isotonic.

In some embodiments, the pharmaceutical composition may be an estrogenic composition. The estrogenic composition comprises an estrogenically effective amount of an ERβ-selective compound, or pharmaceutically acceptable salt thereof, or combination of ERβ-selective compounds as described herein and an additional ingredient. The additional ingredient may be an excipient. The excipient may comprise at least one compound that does not occur naturally with an estrogenic compound in nature. In particular, the excipient may comprise at least one compound that does not naturally occur with an ERβ-compound or combination of ERβ-compounds as described herein in humans. In some embodiments the excipient may comprise at least one compound other than water. In some embodiments, the additional compound may be a salt or other ingredient at a concentration sufficient for the composition to be isotonic. In some embodiments, the additional ingredient may be a flavor or sweetener not found with an ERβ-compound or combination of ERβ-compounds as described herein in nature. In some embodiments, the estrogenic composition may be sterile, pyrogen free, and/or isotonic.

Pharmaceutically acceptable salts may be any salt of an estrogenic compound disclosed herein having suitable solubility in an aqueous solvent of appropriate pH. Remington's, 20th Ed., published 2000, pp. 704-719 provides methods for determining appropriate pharmaceutically acceptable salts. For example, suitable salts may be selected from Table 38-2, p. 704 of Remington's. The pharmaceutically acceptable salt may be prepared by dissolving the estrogenic compound in a suitable solvent and adding a suitable acid or base, or suitable counter-acid or counter-base, as the case may be, to the solution, and separating the salt form of the estrogenic compound from the solution.

Pharmaceutical compositions, in particular estrogenic compositions, may be formulated for a variety of routes of administration, such as oral, intranasal, intrapulmonary (e.g., for inhalation), intravenous, subcutaneous, transdermal, sublingual, buccal, intraperitoneal, or intrathecal administration. Pharmaceutical compositions may comprise one or more enhancers to assist in the transport of an ERβ-compound or combination of ERβ-compounds as described herein against one or more external or internal physiological barriers, such as a pulmonary epithelial barrier or a blood brain barrier.

Suitable pharmaceutically acceptable excipients may include the following types of excipients: diluents, lubricants, binders, disintegrants, fillers, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents.

### Estrogenically Effective Doses

The effective dose of an ERβ-selective compound, or pharmaceutically acceptable salt thereof, or combination of ERβ-selective compounds or pharmaceutically acceptable salts may vary depending upon a variety of factors, including the route of administration, the age and condition of the patient in need of estrogenic treatment, etc. In general, an ERβ-selective compound, or pharmaceutically acceptable salt thereof, or combination of ERβ-selective compounds or pharmaceutically acceptable salts as described herein is effective in vitro at nanomolar or micromolar concentrations. Effective daily doses of an ERβ-selective compound, or pharmaceutically acceptable salt thereof, or combination of ERβ-selective compounds or pharmaceutically acceptable salts as described herein may be in the range of 0.01 mg to 1000 mg per day. The effective daily dose may be divided into two or more divided doses, e.g., 1, 2, 3, 4, 5, 6, or more divided doses. Where a pharmaceutical composition is administered as an infusion, the effective daily dose may be administered as a continuous infusion over a course of hours, e.g., 1-24 hours. An effective dose may be similar to that of a an ERβ-selective compound, or pharmaceutically acceptable salt thereof, or combination of ERβ-selective compounds or pharmaceutically acceptable salts as described herein, but may be scaled to account for the greater molecular weight of the compared to an ERβ-selective compound or combination of ERβ-selective compounds as described herein at the compound's relative bioactivity, pharmacokinetics and pharmacodynamics, which one of skill in the art knows how to determine by art-recognized methods.

One skilled in art of pharmaceutical formulation and compounding possesses the knowledge and skill to select suitable pharmaceutically acceptable carriers and excipients in appropriate amounts for the use with an ERβ-compound or combination of ERβ-compounds as described herein. In addition, there are a number of resources available those skilled in the art, which describe pharmaceutically acceptable carriers and excipients and may be useful in selecting suitable pharmaceutically acceptable carriers and excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The compositions of estrogenic compounds as described herein may be prepared using techniques and methods known to those skilled in the art. Some methods commonly used in the art are described in Remington's Pharmaceutical Sciences, 20th Ed., (Mack Publishing Company (2000)).

In some embodiments, estrogenic compositions may comprise an ERβ-compound or combination of ERβ-compounds as described herein and one or more pharmaceutically acceptable carriers or excipients. The composition may be prepared and packaged in bulk form wherein an effective amount of a compound of the disclosure can be extracted and then given to a subject, such as with powders or syrups. Alternatively, the composition may be prepared and packaged in unit dosage form wherein each physically discrete unit contains an effective amount of an ERβ-compound or combination of ERβ-compounds as described herein.

An ERβ-compound or combination of ERβ-compounds as described herein, and a pharmaceutically acceptable carrier or excipient(s), may be formulated into a dosage form adapted for administration to a subject by a desired route of administration. For example, dosage forms include those adapted for (1) oral administration, such as tablets, capsules, caplets, pills, troches, powders, syrups, elixirs, suspensions, solutions, emulsions, sachets, and cachets; and (2) parenteral administration, such as sterile solutions, suspensions, and powders for reconstitution. Suitable pharmaceutically acceptable carriers or excipients may vary depending upon the particular dosage form chosen. In addition, suitable pharmaceutically acceptable carriers or excipients may be chosen for a particular function that they may serve in the composition. For example, certain pharmaceutically acceptable carriers or excipients may be chosen for their ability to facilitate the production of uniform dosage forms. Certain pharmaceutically acceptable carriers or excipients may be chosen for their ability to facilitate the production of stable dosage forms. Certain pharmaceutically acceptable carriers or excipients may be chosen for their ability to facilitate the carrying or transporting of a compound disclosed herein, once administered to the subject, from one organ or portion of the body to another organ or another portion of the body. Certain pharmaceutically acceptable carriers or excipients may be chosen for their ability to enhance patient compliance.

In some embodiments, estrogenic an ERβ-compound or combination of ERβ-compounds as described herein compositions may be formulated for parenteral administration. Compositions adapted for parenteral administration include aqueous and non-aqueous sterile injection solutions which may contain antioxidants, buffers, bacteriostats, and solutes that render the formulation isotonic with the blood of the intended recipient; and aqueous and non-aqueous sterile suspensions that may include suspending agents and thickening agents. The compositions may be presented in unit-dose or multi-dose containers, for example sealed ampoules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets. Parenteral formulations may be sterile, pyrogen-free, or both. Parenteral formulations may be isotonic.

### Oral

The estrogenic composition may be an oral estrogenic composition comprising an ERβ-selective compound, or pharmaceutically acceptable salt thereof, or combination of ERβ-selective compounds or pharmaceutically acceptable salts as described herein and at least one excipient suitable for oral administration. The at least one excipient suitable for oral administration may comprise a compound that does not occur naturally with a ERβ-compound or combination of ERβ-compounds as described herein in nature. The at least one excipient suitable for oral administration may comprise at least one compound other than water. Various dosage forms may be prepared, such as tablets, capsules, caplets, troches, powders, emulsions, sachets, cachets, gel capsules, elixirs, pills, oral sprays, chewable tablets, sublingual tablets, films, or sprays, or buccal films or sprays.

In some embodiments, ERβ-compound or combination of ERβ-compounds as described herein may be formulated as a solid oral dosage form, such as a tablet or capsule comprising an effective amount of a compound of the disclosure and a diluent or filler. Suitable diluents and fillers include lactose, sucrose, dextrose, mannitol, sorbitol, starch (e.g., corn starch, potato starch, and pre-gelatinized starch), cellulose and its derivatives, (e.g., microcrystalline cellulose), calcium sulfate, and dibasic calcium phosphate. The oral solid dosage form may further comprise a binder. Suitable binders include starch (e.g., corn starch, potato starch, and pre-gelatinized starch) gelatin, acacia, sodium alginate, alginic acid, tragacanth, guar gum, povidone, and cellulose and its derivatives (e.g., microcrystalline cellulose). The oral solid dosage form may further comprise a disintegrant. Suitable disintegrants include crospovidone, sodium starch glycolate, croscarmellose, alginic acid, and sodium carboxymethyl cellulose. The oral solid dosage form may further comprise a lubricant. Suitable lubricants include stearic acid, magnesium stearate, calcium stearate, and talc.

Where appropriate, dosage unit formulations for oral administration can be microencapsulated. The compositions can also be prepared to prolong or sustain the release as, for example, by coating or embedding particulate material in polymers, wax, or the like.

An ERβ-compound or combination of ERβ-compounds as described herein may also be combined with soluble polymers as targetable drug carriers. Such polymers can include polyvinylpyrrolidone, pyrancopolymer, polyhydroxypropylmethacrylamidephenol, polyhydroxy-ethylaspartam-idephenol, or polyethylene-oxidepolylysine substituted with palmitoyl residues. Furthermore, an ERβ-compound or combination of ERβ-compounds as described herein may be combined with a class of biodegradable polymers useful in achieving controlled release of a drug, for example polylactic acid, polepsilon caprolactone, polyhydroxy butyric acid, polyorthoesters, polyacetals, polydihydropyrans, polycyanacrylates and cross-linked or amphipathic block copolymers of hydrogels.

In some embodiments, an ERβ-compound or combination of ERβ-compounds as described herein may be formulated in a liquid oral dosage form. Oral liquids such as solution, syrups and elixirs can be prepared in dosage unit form so that a given quantity contains a predetermined amount of a compound disclosed herein. Syrups can be prepared by dissolving the compound of the disclosure in a suitably flavored aqueous solution, while elixirs are prepared through the use of a non-toxic alcoholic vehicle. Suspensions can be formulated by dispersing a compound disclosed herein in a non-toxic vehicle. Solubilizers and emulsifiers such as ethoxylated isostearyl alcohols and polyoxy ethylene sorbitol ethers, preservatives, flavor additives such as peppermint oil or other natural sweeteners or saccharin or other artificial sweeteners and the like can also be added.

### Intranasal

The estrogenic composition may be an intranasal estrogenic composition comprising an ERβ-compound or combination of ERβ-compounds as described herein and at least one excipient suitable for intranasal administration. The at least one excipient suitable for intranasal administration may comprise at least one compound other than water. For example, the intranasal estrogenic composition may comprise one or more penetration enhancers, which increase absorption of an ERβ-compound or combination of ERβ-compounds as described herein across the mucosa and/or increase bioavailability. In some embodiments, penetration enhancers may include mucolytic agents, degradative enzyme inhibitors and compounds which increase permeability of the mucosal cell membranes. Whether a given compound is an “enhancer” can be determined by comparing two formulations comprising a non-associated, small polar molecule as the drug, with or without the enhancer, in an in vivo or good model test and determining whether the uptake of the drug is enhanced to a clinically significant degree. The enhancer should not produce any problems in terms of chronic toxicity because in vivo the enhancer should be non-irritant and/or rapidly metabolized to a normal cell constituent that does not have any significant irritant effect. In some embodiments, the penetration enhancer may be an alkyl glycoside, e.g., an alkyl glycoside disclosed in U.S. Pat. No. 5,661,130, which is incorporated herein by reference in its entirety. One skilled in the art recognizes the need to achieve a suitable hydrophile-lipophile balance (HLB) number, which may be determined as disclosed in U.S. Pre-Grant Publication No. US2009/0047347, which is incorporated herein by reference in its entirety.

Intranasal estrogenic compositions of an ERβ-compound or combination of ERβ-compounds as described herein may also include flavors or scents to cover the taste of an ERβ-compound or combination of ERβ-compounds as described herein. Intranasal compositions may also include isotonizing agents to make the composition isotonic. Intranasal estrogenic compositions of an ERβ-compound or combination of ERβ-compounds as described herein may also include stabilizing agents.

### Intrapulmonary

The estrogenic composition may be an intrapulmonary estrogenic composition comprising an ERβ-compound or combination of ERβ-compounds as described herein and at least one excipient suitable for intranasal administration. The at least one excipient suitable for intrapulmonary administration may comprise at least one compound other than water. For example, the intrapulmonary composition may comprise one or more penetration enhancers, which increase the ability of an ERβ-compound or combination of ERβ-compounds as described herein to cross the pulmonary epithelia into the blood stream.

Intrapulmonary estrogenic compositions may be administered to the lungs by inhalation, e.g., using an insufflator, aerosol inhaler, or a conventional or high efficiency nebulizer.

High efficiency nebulizers are inhalation devices that comprise a microperforated membrane through which a liquid solution is converted through electrical or mechanical means into aerosol droplets suitable for inhalation. High efficiency nebulizers can deliver a large fraction of a loaded dose to a patient. In some embodiments, the high efficiency nebulizer may also utilize one or more actively or passively vibrating microperforated membranes. In some embodiments, the high efficiency nebulizer may comprise one or more oscillating membranes. In some embodiments, the high efficiency nebulizer may comprise a vibrating mesh or plate with multiple apertures and optionally a vibration generator with an aerosol mixing chamber. In some such embodiments, the mixing chamber may function to collect (or stage) the aerosol from the aerosol generator.

In some embodiments, the high efficiency nebulizer may achieve lung deposition (deposited lung dose) of at least about 10% based on the nominal dose of an ERβ-compound or combination of ERβ-compounds as described herein.

In some embodiments, the high efficiency nebulizer provides an ERβ-compound or combination of ERβ-compounds as described herein lung deposition (deposited lung dose) of at least about 5% based on the nominal dose of an ERβ-compound or combination of ERβ-compounds as described herein.

In accordance with the invention, in some embodiments, a nebulizer, such as a high efficiency nebulizer may be adapted or adaptable to operate in conjunction with a unit dosage form, such as an ampule or vial, which contains a single dose of an ERβ-compound or combination of ERβ-compounds as described herein for estrogenic therapy. The unit dosage form comprises a container that contains an inhalation solution comprising an ERβ-compound or combination of ERβ-compounds as described herein. The container is adapted to cooperate with the high efficiency nebulizer device in such a way as to permit administration of the nominal dose of the inhalation solution to a patient in need thereof. In some embodiments, the high efficiency nebulizer and the unit dosage form are configured so that they are useable together, but not with other devices or dosage forms. In some particular embodiments, the unit dosage form is configured such that it fits into a keyhole-like structure in the high efficiency nebulizer but will not operate with other nebulizer devices. In such embodiments, the high efficiency nebulizer is configured such that it will accept and properly operate with the unit dosage form containing an ERβ-compound or combination of ERβ-compounds as described herein, but not with other dosage forms.

Suitable high efficiency nebulizers with perforated membranes are disclosed in U.S. Pat. Nos. 6,962,151, 5,152,456, 5,261,601, and 5,518,179, each of which is hereby incorporated by reference in its entirety. Suitable high efficiency nebulizers contain oscillatable membranes. Features of these high efficiency nebulizers are disclosed in U.S. Pat. Nos. 7,252,085; 7,059,320; 6,983,747, each of which is hereby incorporated by reference in its entirety.

Commercial high efficiency nebulizers are available from: PARI (Germany) under the trade name eFlow®; Aerogen, Ltd. (Ireland) under the trade names AeroNeb® Go and AeroNeb® Pro, AeroNeb® Solo, and other nebulizers utilizing the OnQ® nebulizer technology; Respironics (Murrysville, Calif.) under the trade names I-Neb®; Omron (Bannockburn, Ill.) under the trade name Micro-Air®; Activaero (Germany) under the trade name Akita®, and AerovectRx (Atlanta, Ga.) under the trade name AerovectRx®.

Conventional nebulizers include, for example jet nebulizers or ultrasonic nebulizers. Jet nebulizers generally utilize compressors to generate compressed air, which breaks the liquid medication into small breathable droplets, which form an aerosolized (atomized) mist. In some of these embodiments, when the patient breathes in, a valve at the top opens, which then allows air into the apparatus, thereby speeding up the mist generation; when the patient breathes out, the top valve closes, thereby slowing down the mist generation while simultaneously permitting the patient to breathe out through the opening of a mouthpiece flap.

Some conventional nebulizers are disclosed in U.S. Pat. Nos. 6,513,727, 6,513,519, 6,176,237, 6,085,741, 6,000,394, 5,957,389, 5,740,966, 5,549,102, 5,461,695, 5,458,136, 5,312,046, 5,309,900, 5,280,784, and 4,496,086, each of which is hereby incorporated by reference in its entirety.

Commercial conventional nebulizers are available from: PARI (Germany) under the trade names PARI LC Plus®, LC Star® and PARI-Jet® A & H Products, Inc. (Tulsa, Okla.) under the trade name AquaTower®; Hudson RCI (Temecula, Calif.) under the trade name AVA-NEB®, Intersurgical, Inc. (Liverpool, N.Y.) under the trade name Cirrus®; Salter Labs (Arvin, Calif.) under the trade name Salter 8900®; Respironics (Murrysville, Pa.) under the trade name Sidestream® Bunnell (Salt Lake City, Utah) under the trade name Whisper Jet®; Smiths-Medical (Hyth Kent, UK) under the trade name Downdraft®, and DeVilbiss (Somerset, Pa.) under the trade name DeVilbiss®.

### Intravenous

The estrogenic composition may be an intravenous estrogenic composition comprising an ERβ-compound or combination of ERβ-compounds as described herein and at least one excipient suitable for intravenous administration. The at least one excipient suitable for intravenous administration may comprise at least one compound other than water. Intravenous compositions of an ERβ-compound or combination of ERβ-compounds as described herein are parenteral compositions intended for intravenous administration by injection or infusion. They may contain one or more isotonizing agents to make the compositions isotonic. They may be, and generally are, sterile, pyrogen free, or both.

### Subcutaneous

The estrogenic composition may be a subcutaneous estrogenic composition comprising an ERβ-compound or combination of ERβ-compounds as described herein and at least one excipient suitable for subcutaneous administration. The at least one excipient suitable for subcutaneous administration may comprise at least one compound other than water. Subcutaneous compositions of an ERβ-compound or combination of ERβ-compounds as described herein are parenteral compositions intended for injection under the skin. They may contain one or more isotonizing agents to make the compositions isotonic. They may be, and generally are, sterile, pyrogen free, or both.

### Transdermal

The estrogenic composition may be a transdermal estrogenic composition comprising an ERβ-compound or combination of ERβ-compounds as described herein and at least one excipient suitable for transdermal administration. The at least one excipient suitable for transdermal administration may comprise at least one compound other than water. For example, the transdermal estrogenic composition may comprise one or more penetration enhancers, which increase the ability of an ERβ-compound or combination of ERβ-compounds as described herein to cross the dermis into the blood stream. In addition, the transdermal composition may be delivered by a biasing mechanism, such as an iontophoresis device.

### Sublingual or Buccal

The estrogenic composition may be a sublingual or buccal estrogenic composition comprising an ERβ-compound or combination of ERβ-compounds as described herein and at least one excipient suitable for sublingual or buccal administration. The at least one excipient suitable for sublingual or buccal administration may comprise at least one compound other than water.

### Intraperitoneal

The estrogenic composition may be an intraperitoneal estrogenic composition comprising an ERβ-compound or combination of ERβ-compounds as described herein and at least one excipient suitable for intraperitoneal administration. The at least one excipient suitable for intraperitoneal administration may comprise at least one compound other than water. Intraperitoneal estrogenic compositions of an ERβ-compound or combination of ERβ-compounds as described herein are parenteral compositions intended for administration to the peritoneum by injection or infusion. They may contain one or more isotonizing agents to make the compositions isotonic. They may be, and generally are, sterile, pyrogen free, or both.

### Intrathecal or Intracranioventricular

The estrogenic composition may be an intrathecal or intracranioventricular estrogenic composition comprising an ERβ-compound or combination of ERβ-compounds as described herein and at least one excipient suitable for intrathecal or intracranioventricular administration. The at least one excipient suitable for intrathecal or intracranioventricular administration may comprise at least one compound other than a compound that occurs naturally with an ERβ-compound or combination of ERβ-compounds as described herein in nature, e.g., water. Intrathecal or intracranioventricular estrogenic compositions of an ERβ-compound or combination of ERβ-compounds as described herein are parenteral compositions intended for administration into the cerebrospinal fluid administration by injection or infusion. They may contain one or more isotonizing agents to make the compositions isotonic. They may be, and generally are, sterile, pyrogen free, or both.

### Other Routes of Administration

Although there have been shown and described preferred embodiments of the compositions and methods described herein, it will be readily apparent to those skilled in the art that modifications may be made thereto which do not exceed the scope of the appended claims. Therefore, the scope of the described compositions and methods is only to be limited by the following claims.

### Estrogenic Methods

Some embodiments described herein provide a compound of Table 1, or a pharmaceutically acceptable salt thereof, or a pharmaceutical composition described herein, for use in treatment of a condition or disease state in a patient in need of treatment with a nuclear receptor reprogramming compound. In some embodiments, the condition or disease state is a development condition, a menstruation condition, a fertility condition, a gynecological disease, an autoimmune disorder, a menopause disorder, an aging disorder, or cancer. In some embodiments, the condition or disease state is Turner Syndrome, Kallman Syndrome, congenital primary amenorrhea, a childhood neuropsychiatric disorder, dysmenorrhea, amenorrhea, menorrhagia, estrogen-induced deep vein thrombosis, pulmonary embolism, conception, fetal implantation, spontaneous abortion, preterm labor, endometriosis, polycystic ovarian syndrome, rheumatoid arthritis, scleroderma, Sjogren's syndrome, Hashimoto's thyroiditis, multiple sclerosis, irritable bowel syndrome, ulcerative colitis, Crone's disease, vasomotor symptoms of menopause and perimenopause, insomnia, nighttime wakening, mood swings, vulvovaginal atrophy, vaginal dryness, dyspareunia, menopausal weight gain and obesity, osteoporosis, type-2 diabetes, estrogen-induced deep vein thrombosis and pulmonary embolism, Alzheimer's disease, early dementia, breast cancer, uterine cancer, ovarian cancer, prostate cancer, and non-small cell lung cancer.

Some embodiments described herein provide for use of a compound of Table 1, or a pharmaceutically acceptable salt thereof, for manufacture of a medicament for use in treatment of a condition or disease in a patient in need of treatment with a nuclear receptor reprogramming compound. In some embodiments, the condition or disease state is a development condition, a menstruation condition, a fertility condition, a gynecological disease, an autoimmune disorder, a menopause disorder, an aging disorder, or cancer. In some embodiments, the condition or disease state is Turner Syndrome, Kallman Syndrome, congenital primary amenorrhea, a childhood neuropsychiatric disorder, dysmenorrhea, amenorrhea, menorrhagia, estrogen-induced deep vein thrombosis, pulmonary embolism, conception, fetal implantation, spontaneous abortion, preterm labor, endometriosis, polycystic ovarian syndrome, rheumatoid arthritis, scleroderma, Sjogren's syndrome, Hashimoto's thyroiditis, multiple sclerosis, irritable bowel syndrome, ulcerative colitis, Crone's disease, vasomotor symptoms of menopause and perimenopause, insomnia, nighttime wakening, mood swings, vulvovaginal atrophy, vaginal dryness, dyspareunia, menopausal weight gain and obesity, osteoporosis, type-2 diabetes, estrogen-induced deep vein thrombosis and pulmonary embolism, Alzheimer's disease, early dementia, breast cancer, uterine cancer, ovarian cancer, prostate cancer, and non-small cell lung cancer.

Some embodiments described herein provide a method of treating a patient in need of such treatment, comprising administering to the patient an effective amount of a compound of Table 1, or a pharmaceutically acceptable salt thereof, or a pharmaceutical composition described herein. In some embodiments, the condition or disease state is a development condition, a menstruation condition, a fertility condition, a gynecological disease, an autoimmune disorder, a menopause disorder, an aging disorder, or cancer. In some embodiments, the condition or disease state is Turner Syndrome, Kallman Syndrome, congenital primary amenorrhea, a childhood neuropsychiatric disorder, dysmenorrhea, amenorrhea, menorrhagia, estrogen-induced deep vein thrombosis, pulmonary embolism, conception, fetal implantation, spontaneous abortion, preterm labor, endometriosis, polycystic ovarian syndrome, rheumatoid arthritis, scleroderma, Sjogren's syndrome, Hashimoto's thyroiditis, multiple sclerosis, irritable bowel syndrome, ulcerative colitis, Crone's disease, vasomotor symptoms of menopause and perimenopause, insomnia, nighttime wakening, mood swings, vulvovaginal atrophy, vaginal dryness, dyspareunia, menopausal weight gain and obesity, osteoporosis, type-2 diabetes, estrogen-induced deep vein thrombosis and pulmonary embolism, Alzheimer's disease, early dementia, breast cancer, uterine cancer, ovarian cancer, prostate cancer, and non-small cell lung cancer.

In light of the disclosure herein, one skilled in the art understands how to determine an estrogenically effective amount of an ERβ-compound or combination of ERβ-compounds as described herein. Generally, an estrogenically effective amount, a therapeutically effective amount, or a prophylactically effective amount, of an ERβ-compound or combination of ERβ-compounds as described herein may be determined, e.g., by inference from in vitro testing. One skilled in the art understands that an effective dose may be inferred from the in vitro half maximal modulating (inhibitory or activating) concentration of an ERβ-compound or combination of ERβ-compounds as described herein. One skilled in the art understands that the effective dose in human patients will depend on the route of administration, the pharmacokinetics, etc. Taking these factors into consideration, an estrogenically effective dose of an ERβ-compound or combination of ERβ-compounds as described herein may be in the range of 0.1 mg/kg to 150 mg/kg, e.g. 0.1 mg/kg to 1 mg/kg, 0.5 mg/kg to 5 mg/kg, 1 mg/kg to 10 mg/kg, 5 mg/kg to 50 mg/kg, 10 mg/kg to 100 mg/kg, or 50 mg/kg to 150 mg/kg; an effective daily dose of an ERβ-compound or combination of ERβ-compounds as described herein may be some multiple of any of the values within these ranges, e.g. one to six (1 to 6) times the values within these ranges.

### Transitional Phrases

In some embodiments, descriptions of the compositions and methods described herein using the transitional word “comprising” indicates that the compositions or methods are “open” to additional ingredients, components or steps. It is intended that “comprising” subsume the more limiting transitional phrases “consisting essentially of” and “consisting of.” Thus, disclosure herein of matter following the transitional phrase “comprising” also fully discloses the same following the transitional phrases “consisting essentially of” or “consisting of.” The transitional phrase “consisting essentially of,” is of intermediate effect, indicating that the subject matter that follows consists only of the recited elements and such additional matter as does not materially affect the novel and basic properties of the claim or claim element. The transitional phrase “consisting of,” indicates that the subject matter that follows is limited to the recited steps or ingredients and is closed to other steps or ingredients not recited. Where a transitional phrase appears within a clause or a sub-clause following another transitional phrase, it is intended that the embedded transitional phrase affect only the phrase in which it appears. Where “a” or “an” is recited in the specification or claims, the plural is also intended unless the singular is explicitly recite (e.g., “a single,” “only one,” etc.).

### EXAMPLES

Pharmaceutical compositions and estrogenic methods disclosed herein may be further understood with reference to the following examples.

We propose new drugs to pharmacologically selectively modulate ERβ activity. These drugs are selective ERβ subtype agonists. They do not activate reporter genes like estradiol on the ERα. Instead, this class of compounds produce a selective ERβ activation of reporter genes and regulate genes on various tissue types differently from estradiol.

### Example 1: ERβ-Selective Compounds

Liquiritigenin; Apigenin; Luteolin; Galangin; Naringenin; Calycosin; 6-methoxyluteolin; Nyasol; Wogonin; Broussonin A; 7,4′-Dihydroxyflavone; 6,4′-Dhydroxyflavone; 5,4′-Dihydroxyflavone; 3,4′-Dihydroxyflavone; 7,2′-Dihydroxyflavone; 7,3′-Dihydroxyflavone; Genistein; and Equol were obtained from commercial sources, e.g., Sigma-Aldrich.

Analog 13 (A13) was obtained by the following synthetic method:

Synthesis of (E)-1,3-bis(4-methoxyphenyl) prop-2-en-1-one 2. To a solution of 4-methoxybenzaldehyde 1 (300 mg, 2.2 mmol) in EtOH:H₂O (4 mL, V_{EtOH}:V_{H2O}=3:1) was added 1-(4-methoxyphenyl) ethan-1-one a (330 mg, 2.2 mmol) and KOH (246 mg, 4.4 mmol). The result solution was stirred for 12 h at 0° C.˜rt. The mixture was filtered off to give (E)-1,3-bis(4-methoxyphenyl) prop-2-en-1-one 2 (0.57 g, 96%) as a white solid.

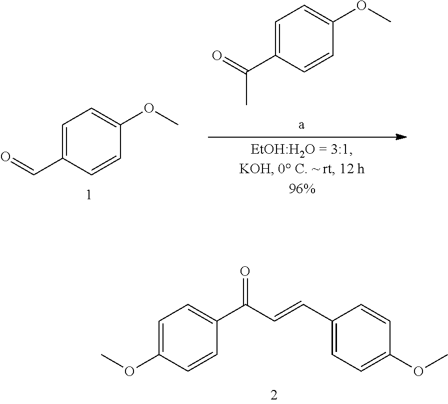

Synthesis of (E)-1,3-bis(4-hydroxyphenyl) prop-2-en-1-one Analog 13. To a solution of (E)-1,3-bis(4-methoxyphenyl) prop-2-en-1-one 2 (200 mg, 0.74 mmol) in dry DCM (5 mL) was added dropwise BBr₃ (0.93 g, 3.73 mmol) at 0° C. under N₂. The resulting mixture was stirred at temperature for 12 h, and then quenched by water at temperature. Finally crude product was purified by flash reversed phase column (MeOH in water=5% to 100%, 60 min) to give N-benzyl-2,3,4-trihydroxybenzamide CAT8511-13 (150 mg, 83%) as a yellow solid. ¹H NMR (400 MHZ, DMSO-d₆) δ 10.30 (s, 1H), 8.03 (d, J=8.7 Hz, 2H), 7.76-7.53 (m, 4H), 6.85 (dd, J=19.7, 8.6 Hz, 4H).

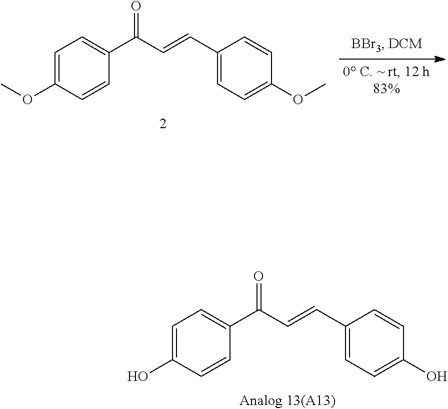

### A10

Synthesis of (E)-3-(3-methoxyphenyl)-1-(2,3,4-trimethoxyphenyl) prop-2-en-1-one 2. To a solution of 1-(2,3,4-trimethoxyphenyl) ethan-1-one 1 (600 mg, 2.8 mmol) in EtOH:H₂O (8 mL, V_{EtOH}:V_{H2O}=3:1) was added 3-methoxybenzaldehyde a (387 mg, 2.8 mmol) and KOH (1600 mg, 28 mmol). The result solution was stirred for 12 h at 0° C.˜rt. The mixture was filtered off to give (E)-3-(3-methoxyphenyl)-1-(2,3,4-trimethoxyphenyl) prop-2-en-1-one 2 (600 mg, 64%) as a white solid.

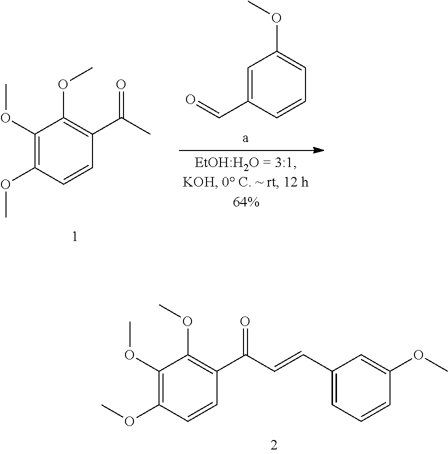

Synthesis of (E)-3-(3-hydroxyphenyl)-1-(2,3,4-trihydroxyphenyl) prop-2-en-1-one A10. To a solution of (E)-3-(3-methoxyphenyl)-1-(2,3,4-trimethoxyphenyl) prop-2-en-1-one 2 (200 mg, 0.61 mmol) in dry DCM (5 mL) was added dropwise BBr₃ (762 mg, 3 mmol) at 0° C. under N₂. The resulting mixture was stirred at temperature for 12 h, and then quenched by water at temperature. Finally crude product was purified by flash reversed phase column (MeOH in water=5% to 100%, 60 min) to give (E)-3-(3-hydroxyphenyl)-1-(2,3,4-trihydroxyphenyl) prop-2-en-1-one A10 (63 mg, 38%) as a yellow solid. ¹H NMR (400 MHZ, DMSO-d₆) δ 13.60 (s, 1H), 10.12 (s, 2H), 8.63 (s, 1H), 7.73 (q, J=9.0, 8.5 Hz, 5H), 6.84 (d, J=8.6 Hz, 2H), 6.43 (d, J=8.9 Hz, 1H).

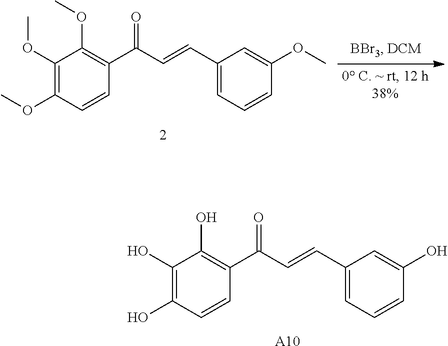

### A13

Synthesis of (E)-1,3-bis(4-methoxyphenyl) prop-2-en-1-one 2. To a solution of 4-methoxybenzaldehyde 1 (300 mg, 2.2 mmol) in EtOH:H₂O (4 mL, V_{EtOH}:V_{H2O}=3:1) was added 1-(4-methoxyphenyl) ethan-1-one a (330 mg, 2.2 mmol) and KOH (246 mg, 4.4 mmol). The result solution was stirred for 12 h at 0° C.˜rt. The mixture was filtered off to give (E)-1,3-bis(4-methoxyphenyl) prop-2-en-1-one 2 (0.57 g, 96%) as a white solid.

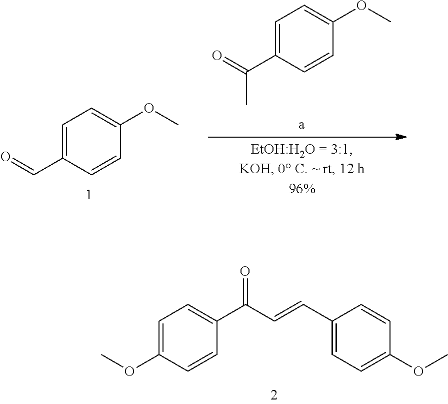

Synthesis of (E)-1,3-bis(4-hydroxyphenyl) prop-2-en-1-one. To a solution of (E)-1,3-bis(4-methoxyphenyl) prop-2-en-1-one 2 (200 mg, 0.74 mmol) in dry DCM (5 mL) was added dropwise BBr₃ (0.93 g, 3.73 mmol) at 0° C. under N₂. The resulting mixture was stirred at temperature for 12 h, and then quenched by water at temperature. Finally crude product was purified by flash reversed phase column (MeOH in water=5% to 100%, 60 min) to give N-benzyl-2,3,4-trihydroxybenzamide A13 (150 mg, 83%) as a yellow solid. ¹H NMR (400 MHZ, DMSO-d₆) δ 10.30 (s, 1H), 8.03 (d, J=8.7 Hz, 2H), 7.76-7.53 (m, 4H), 6.85 (dd, J=19.7, 8.6 Hz, 4H).

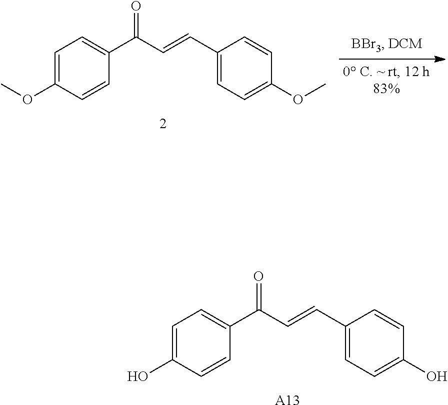

### A15

Synthesis of (E)-3-(4-hydroxyphenyl)-1-phenylprop-2-en-1-one A15. To a solution of 4-hydroxybenzaldehyde 1 (100 mg, 0.82 mmol) in EtOH (5 mL) was added acetophenone a (98 mg, 0.82 mmol) and KOH (92 mg, 1.64 mmol). The result solution was stirred for 12 h at room temperature. The mixture was diluted with EA (50 mL), washed with brine, concentrated under reduced pressure and purified by column chromatography on silica gel with PE/EA=5/1 to give (E)-3-(4-hydroxyphenyl)-1-phenylprop-2-en-1-one A15 (40 mg, 23%) as a yellow solid. ¹H NMR (400 MHZ, Chloroform-d) δ 8.06-7.97 (m, 2H), 7.78 (d, J=15.7 Hz, 1H), 7.62-7.54 (m, 3H), 7.51 (t, J=7.5 Hz, 2H), 7.41 (d, J=15.6 Hz, 1H), 6.89 (d, J=8.6 Hz, 2H).

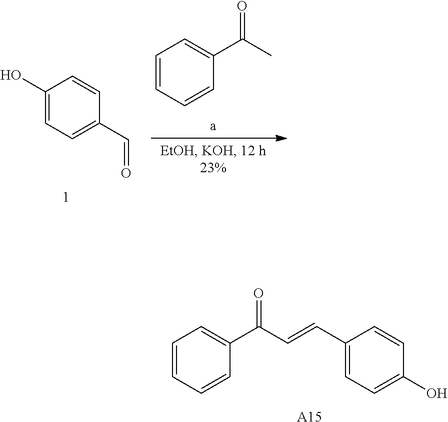

### Example 2: Luciferase Activity in NKG2E-TK-Luc-Transfected U2OS-ERβ Cells with Doxycycline

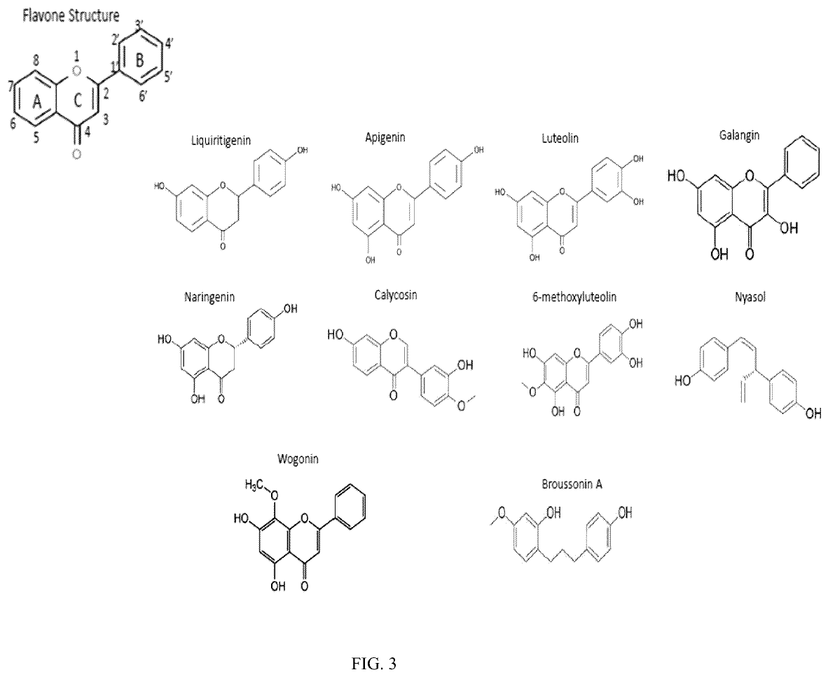

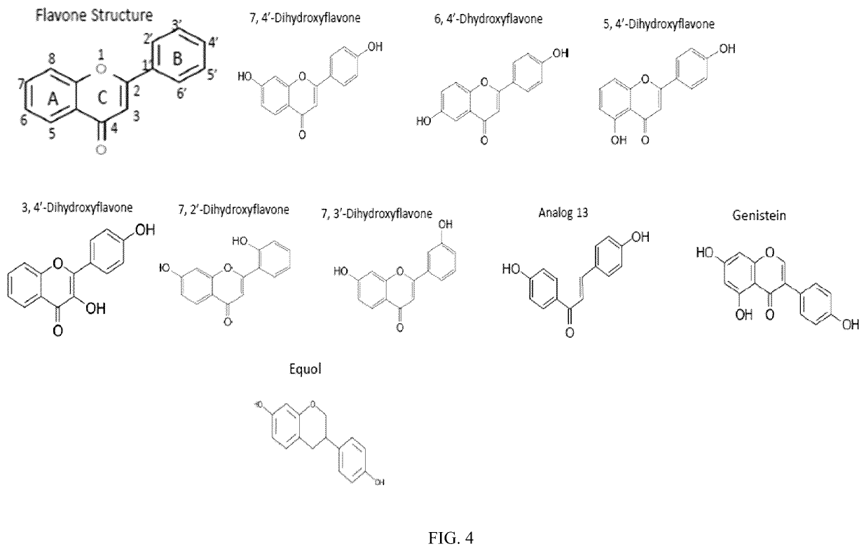

U2OS cells (wild type) were maintained in 5% charcoal-dextran stripped FBS. The cells were transfected with 3 μg of a plasmid containing the ERE upstream of the minimal thymidine kinase luciferase promoter (NKG2E-TK-Luk) and 1 μg of an ERβ expression vector by electroporation as previously described (An et al. 2001). The resulting ERE-TK-Luc/ERβ U2OS cells were incubated for 24 hours in the presence of medium (negative control), E2, and the Test Compounds at the indicated micromolar concentrations (see FIG. 3-4) or E2 (10 nM). As expected, E2 alone activated ERE-TK-Luc. (See U.S. Pat. No. 7,482,029, which is incorporated by reference in its entirety.)

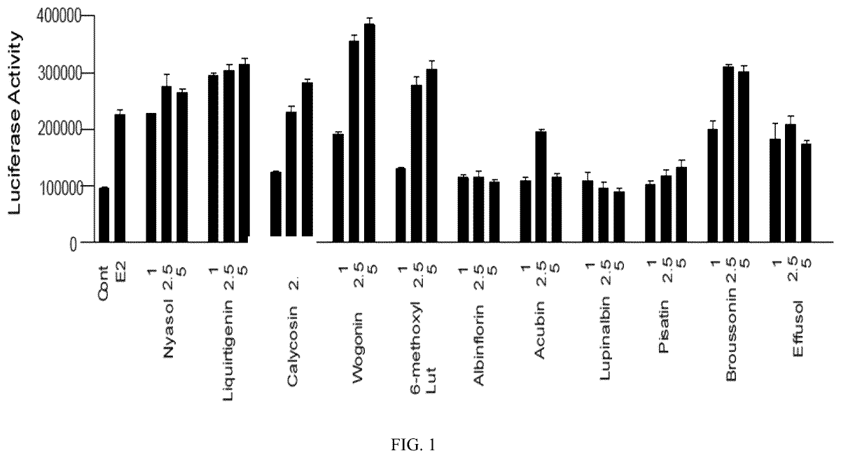

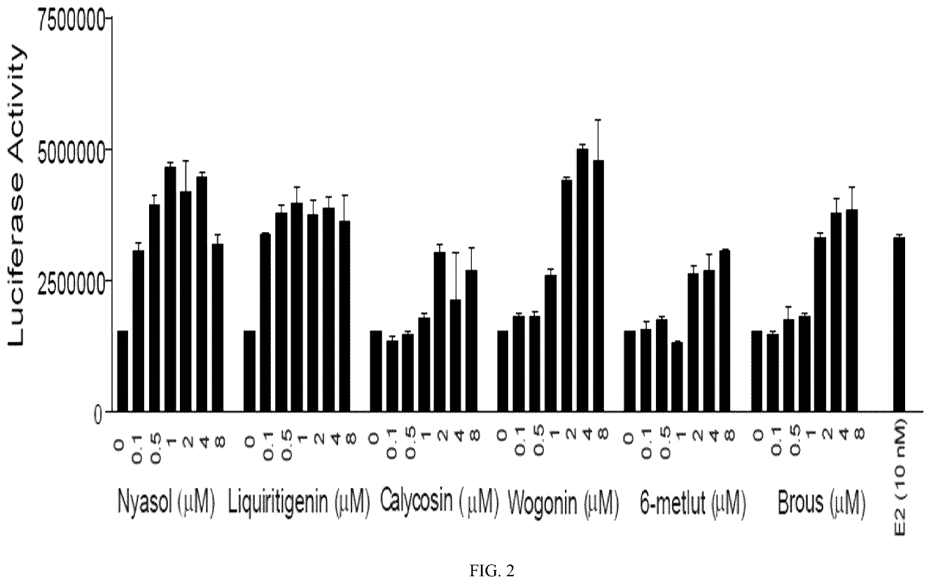

Compounds depicted in FIGS. 3 and 4 were obtained or prepared and the dose response was tested in doxycycline-dependent U2OS cells transfected with NKG2E-TK-Luk and ERβ in the presence of E2 (10 nM) alone in the presence of varying micromolar concentrations of compounds of FIGS. 3 and 4. Luciferase activity for each of the tested compounds was recorded. See FIGS. 1 and 2. As can be seen in FIGS. 1 and 2, several of the compounds showed exceptional ERβ activity.

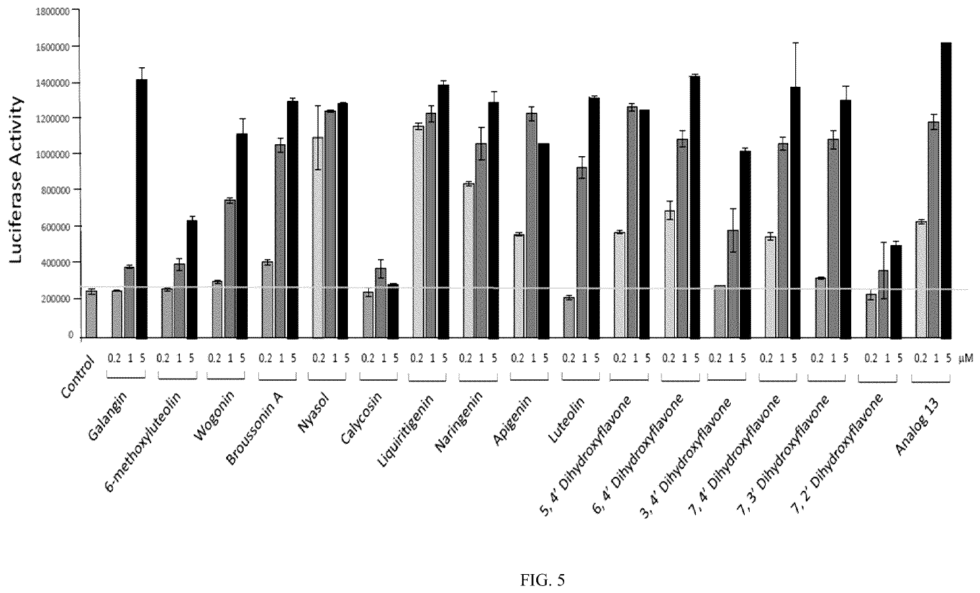

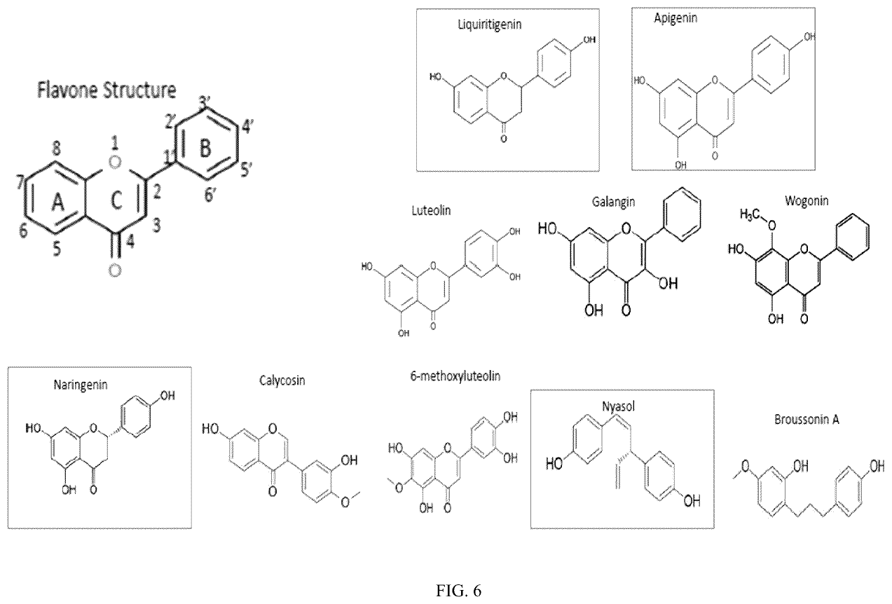

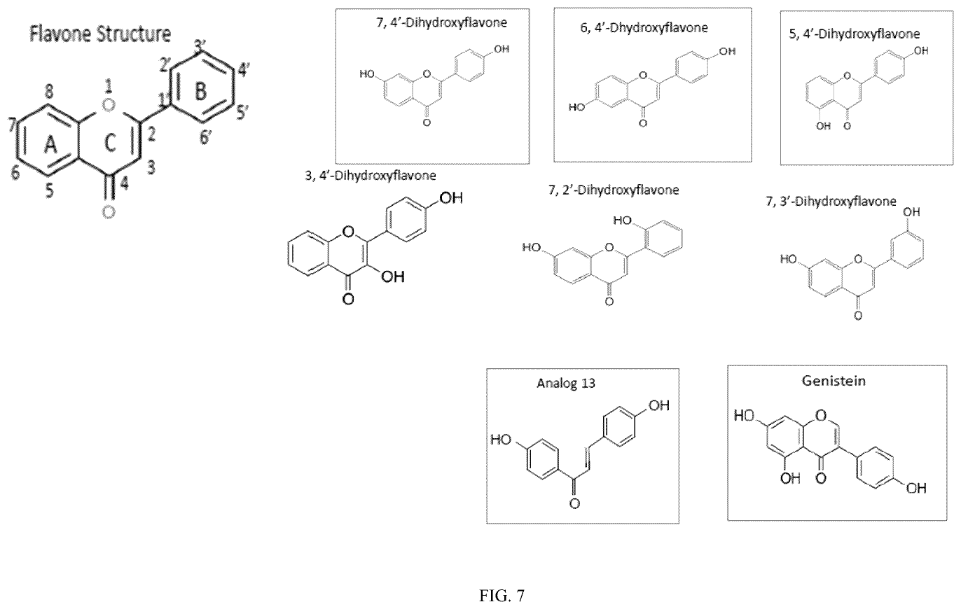

FIG. 5 summarizes dose response activities of ERβ ligands in NKG2E ERE in U2OS-ERβ cells. FIGS. 6 and 7 indicate those tested compounds that activated ERβ at 200 nM concentrations (green boxes). Such compounds, or combinations thereof, including pharmaceutical compositions, are great interest for use in drugs for treatment of symptoms, diseases and disorders described herein.

### Example 3: Dose Response Luciferase Activity of Ligands on the Activation of the NKG2E ERE in U2OS-ERα Cells

U2OS cells (wild type) were maintained in 5% charcoal-dextran stripped FBS. The cells were transfected with 3 μg of a plasmid containing the ERE upstream of the minimal thymidine kinase luciferase promoter (NKG2E-TK-Luk) and 1 μg of an ERα expression vector by electroporation as previously described (An et al. 2001). The resulting ERE-TK-Luc/ERβ U2OS cells were incubated for 24 hours in the presence of medium (negative control), E2, and the Test Compounds at the indicated micromolar concentrations (see FIG. 3-4) or E2 (10 nM). As expected, E2 alone activated ERE-TK-Luc. (See U.S. Pat. No. 7,482,029, which is incorporated by reference in its entirety.)

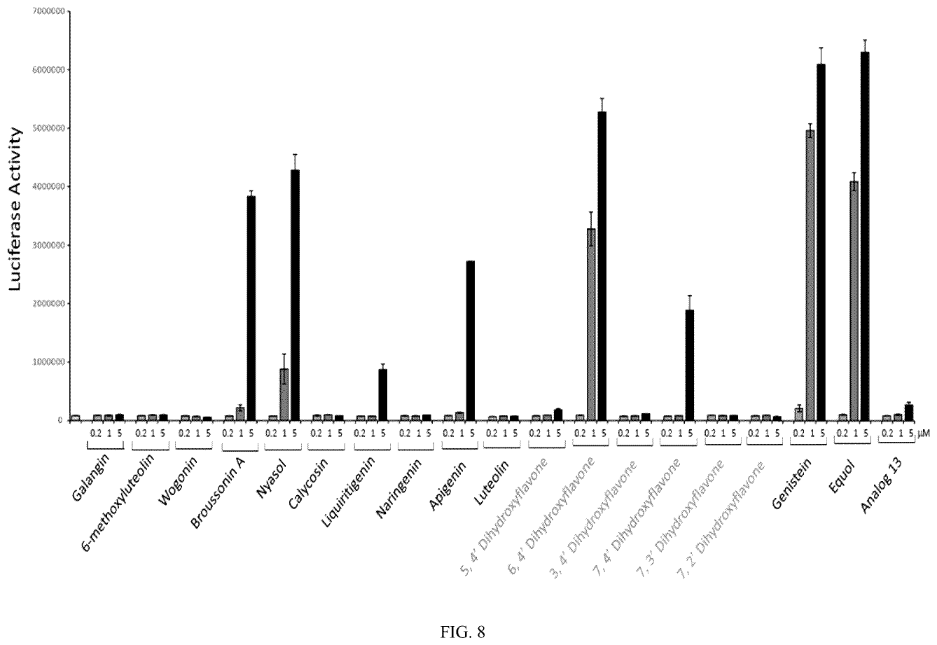

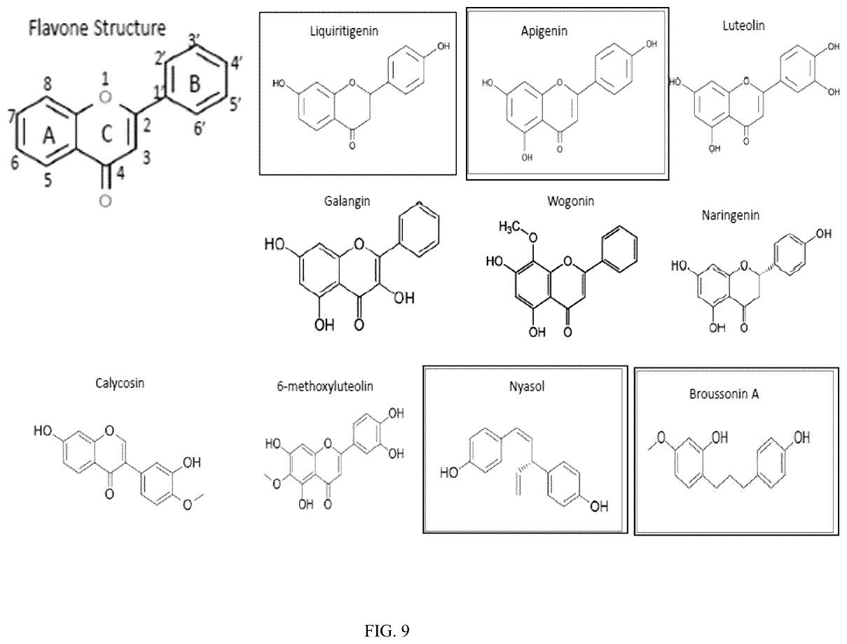

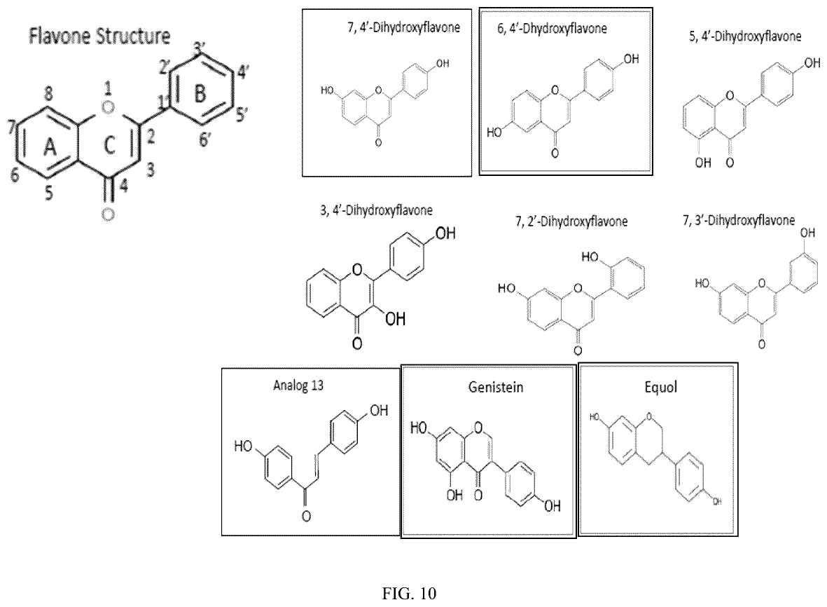

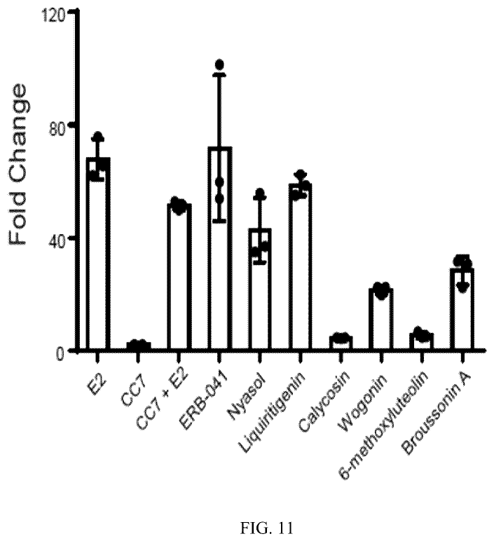

Several of the Test Compounds showed little or no activity in ERα U2OS cells. Liquiritigenin; Apigenin; Luteolin; Galangin; Naringenin; Calycosin; 6-methoxyluteolin; Nyasol; Wogonin; Broussonin A; 7,4′-Dihydroxyflavone; 6,4′-Dhydroxyflavone; 5,4′-Dihydroxyflavone; 3,4′-Dihydroxyflavone; 7,2′-Dihydroxyflavone; 7,3′-Dihydroxyflavone; Genistein; and Equol. See FIGS. 8-10.

### Example 4: Keratin 19 RNA Isolation and Quantitative Real-Time PCR

Total cellular RNA was extracted using the Aurum Total RNA Mini Kit (Bio-Rad Laboratories, Hercules, CA, USA) following the manufacturer's protocol. Reverse transcription reactions were performed using the iScript cDNA Synthesis Kit (Bio-Rad, Hercules, CA, USA) with 1 μm of total RNA according to the manufacturer's protocol. Quantitative reverse transcription-polymerase chain reaction (qRT-PCR) was performed with a Bio-Rad CFX96 Thermal Cycler System using SsoFast EvaGreen Supermix (Bio-Rad, Hercules, CA, USA). The results were analyzed by competitive Ct method (Schmittgen and Livak 2008). The Ct values of the Keratin 19 gene was normalized to the reference gene glyceraldehyde-3-phosphate dehydrogenase (GAPDH) running concurrently to obtain adjusted Ct values (ΔCt). Fold changes were calculated by comparing ΔCt values from various treatments to control samples.

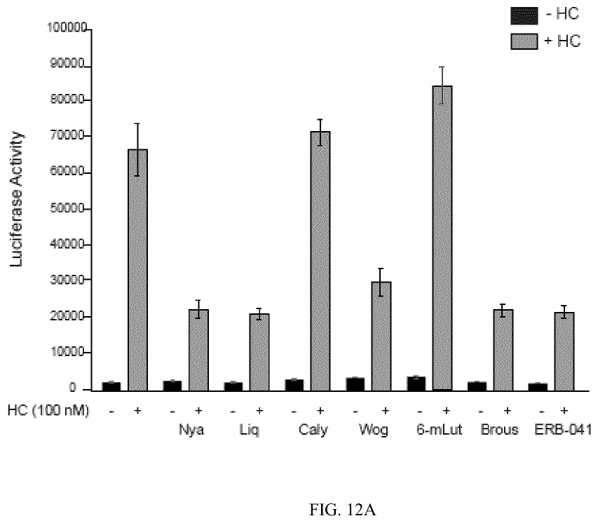

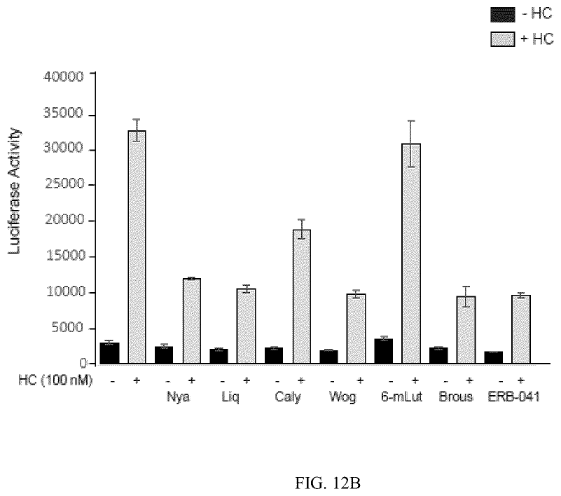

Example 5: Luciferase activity in doxycycline-dependent ERβ-U2OS cells transfected with GRE-TK-Luc and GR in the presence of doxycycline. To understand the activity of the ERβ-active compounds of the invention on GRE, doxycycline-dependent ERβ-U2OS cells transfected with GRE-TK-Luc, GR, and ERβ in the presence of doxycycline were incubated with test compounds (see x-axis label, FIG. 12A) in the absence and presence of hydrocortisone (HC) at 100 nM. The results are set forth in FIG. 12A.

Example 6: Luciferase activity in wild-type (WT) U2OS cells transfected with GRE-TK-Luc, GR and ERβ. To understand the activity of the ERβ-active compounds of the invention on GRE, WT ERβ-U2OS cells transfected with GRE-TK-Luc, GR, and ERβ in the presence of doxycycline were incubated with test compounds (see x-axis label, FIG. 12B) in the absence and presence of hydrocortisone (HC) at 100 nM. The results are set forth in FIG. 12B.

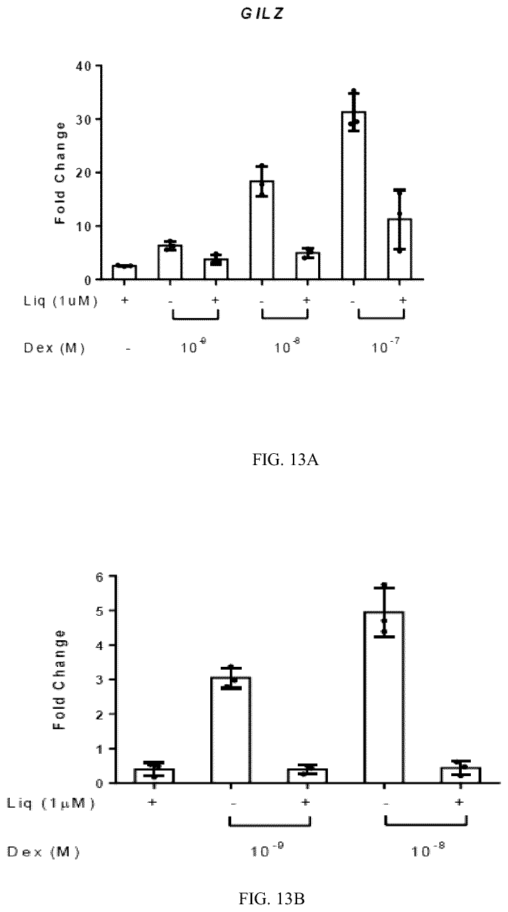

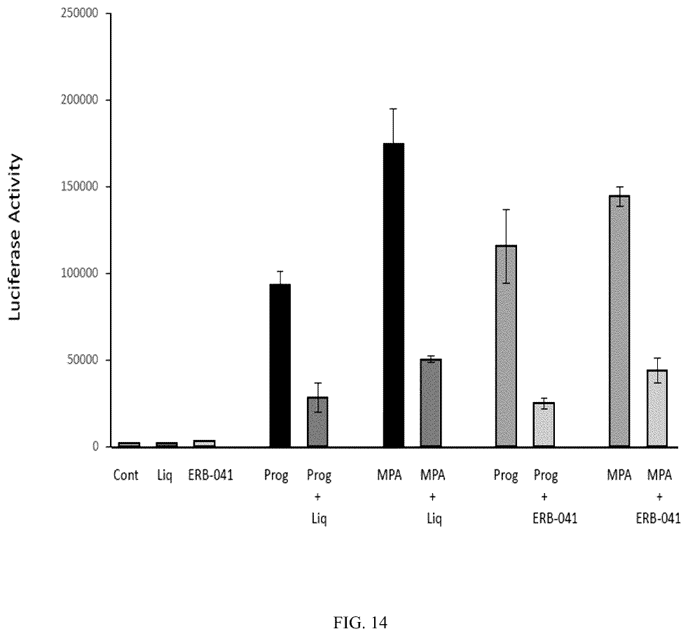

Example 8: GILZ and Ladinin Expression in U2OS cells. Following similar protocols to Example 4, the expression of GILZ and Ladinin were determined in U2OS cells transfected with GRE-TK-Luc, GR and ERβ in the presence of doxycycline. Vehicle, Liquiritigenin and/or Dexamethasone (Dex) were added at the indicated concentrations. See x-axis, FIGS. 13A, 13B. The results are set forth in FIG. 13A (GILZ) and FIG. 13B (Ladinin).

Example 9: Luciferase activity in TAT3-TK-Luc ERβ U2OS cells. U2OS cells were transfected with tyrosine aminotransferase 3 (TAT3)-TK-Luc and ERβ following procedures similar to those described in Example 2. The transfected cells were incubated in the presence of Liquiritigenin or ERβ-41 (Positive control) in the absence of progesterone (Prog) and MPA. The results are shown in FIG. 14.

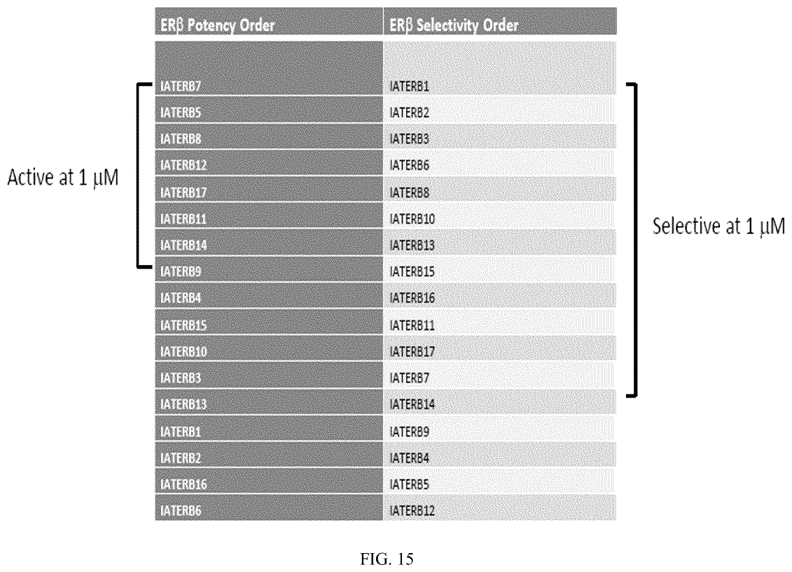

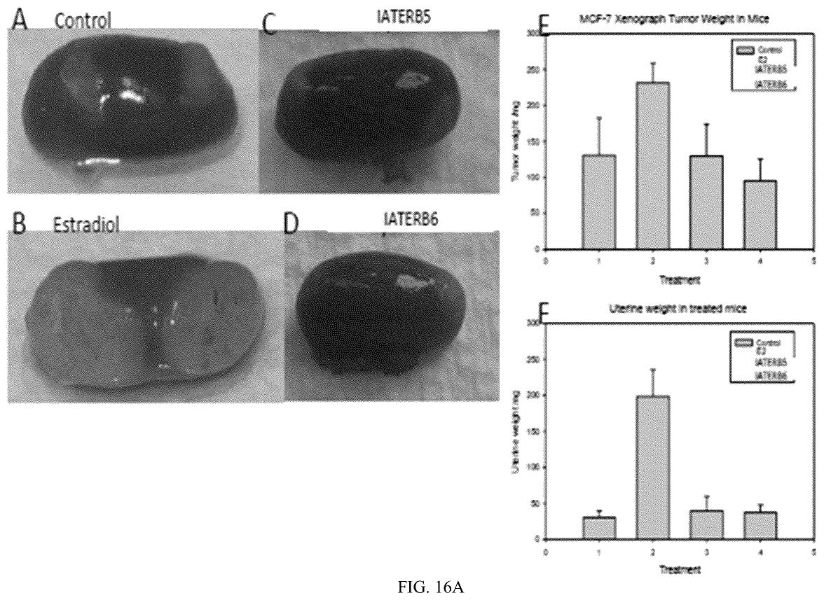

Example 10: Several compounds isolated from MF101 were isolated and tested for ERβ activity. The Table in FIG. 15 ranks these compounds by ERβ potency and selectivity. The names of the compounds are set forth in Table 1, supra.

Example 11: Effects of ERβ compounds on uterine and breast cancer cells. Uterine cancer cells and MCF-7 breast cancer cells were incubated in the presence of vehicle (control), estradiol (E2), IATERB5, IATERB6, and IATERB7. See Table 1, supra, for names of IATERB5-7.

MCF-7 cells were plated at a density of 50,000 cells per well in 6-well tissue culture plates in DMEM/F12 supplemented with 5% stripped FBS. The next day the cells were treated with vehicle or E2 in the absence and presence of 2′,3′,4′-THC or one of the six analogs in FIG. 4 for 7 days. The cells were then detached with trypsin, neutralized with media containing 5% FBS, and resuspended. Appropriate amounts of cell suspension were placed in ISOTON II diluent (Thermo Fisher Scientific, Waltham, MA, USA) and the cell numbers were then measured using a Coulter Counter (Beckman, Brea, CA, USA).

Flow cytometry was performed based on a previously described method (Pan et al. 2016). Briefly, the cells were plated at a density of 500,000 cells per well in 6-well tissue culture dishes in DMEM/F-12 supplemented with 5% stripped FBS for 48 h. The cultured medium was then replaced by serum-free DMEM/F12 for 24 h. The cells were then treated with vehicle, E2 without or with the 2′,3′,4′-THC or one of the six analogs in FIG. 4 for 24 h. The culture medium was then aspirated, and the cells were washed with PBS, detached with trypsin and collected by centrifugation at 1700 rpm for 5 min. The cell pellets were washed with ice cold PBS followed by centrifugation at 1700 rpm for 10 min at room temperature. The cell pellets were resuspended in 500 μL PBS containing 50 μm/mL propidium iodide, 0.1% of triton X-100, 0.1% of sodium citrate, and 10 μm/mL of RNase. The cell suspensions were then analyzed with a BD LSR II Flow Cytometer (BD Biosciences, San Jose, CA, USA) in the Flow Cytometry facility at University of California, Berkeley and the percentage of cells in cell cycle phases were determined by using FlowJo 7.6.5 (FlowJo, LLC, Ashland, OR, USA).

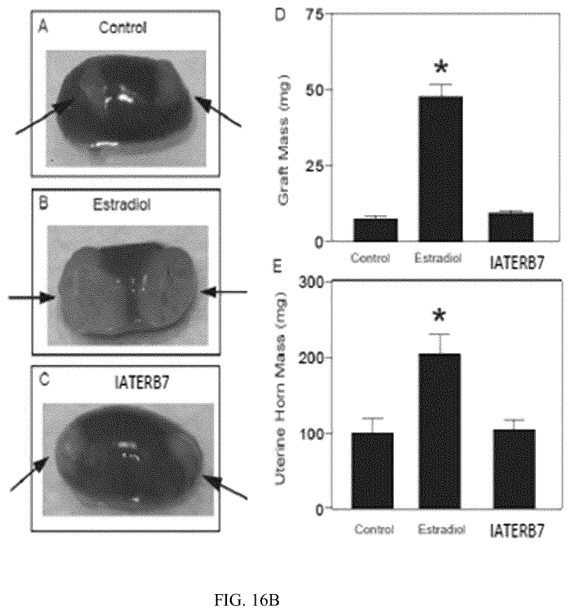

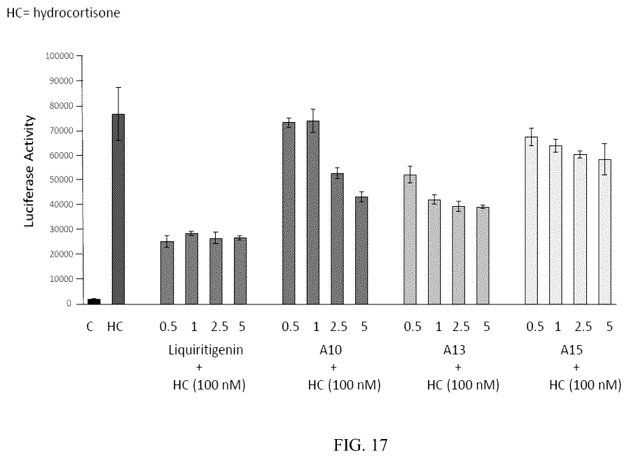

Unlike estradiol, three of the ERβ compounds (IATERB5, IATERB6, and IATERB7) do not increase uterine weight or cause breast cancer tumor formation. See FIG. 16A-B for histology.

Example 12: Luciferase Activity in GRE-TK-Luc-Transfected U2OS-ERβ cells with doxycycline. Doxycycline-dependent U2OS cells were transfected with GRE-TK-Luk, GR, and ERβ in the presence of hydrocortisone (HC, 100 nM) alone or in combination with Liquiritigenin, A10, A13, and A15. Luciferase activity for each of the compounds at 0.5, 1, 2.5 and 5 μM concentration was recorded. Each of the tested compounds attenuated the effect of HC on ERβ activity.

Statistical analysis: All data are presented as the mean±SE or SD from at least biological triplicates. The statistical significance of the difference between two groups was assessed by the Student's t-test. For the data sets consisting of more than two groups, the statistical significance of differences among various groups (treatments) were analyzed by one-way analysis of variance (one-way ANOVA) tests or two-way ANOVA as specified in figure legends. All ANOVA tests were followed by Tukey's or Sidak's multiple comparisons post hoc tests to analyze the significance of differences between any two different treatment groups or control, as indicated in the figure legend. Statistical analysis and graph plotting were performed using GraphPad Prism version 6 (GraphPad Software Inc., San Diego, CA, USA). The statistical significance for the numbers of asterisks in the figures are *p<0.05; **p<0.01, ***p<0.001, and ****p<0.0001.

While a number of embodiments of pharmaceutical compositions and estrogenic methods are described herein, one skilled in the art understand that the examples may be altered to provide other embodiments that utilize an ERβ-compound or combination of ERβ-compounds as described herein compositions and methods described herein. Therefore, it will be appreciated that the scope of this invention is to be defined by the appended claims rather than by the specific embodiments that have been represented by way of example.

What is claimed is:

1. A compound, or a pharmaceutically acceptable salt thereof, wherein the compound is an ERβ-selective estrogenic compound.

2. The compound of claim 1, or a pharmaceutically acceptable salt thereof, wherein the ERβ-selective estrogenic compound is a member of the group consisting of: Liquiritigenin; Apigenin; Luteolin; Galangin; Naringenin; Calycosin; 6-methoxyluteolin; Nyasol; Wogonin; Broussonin A; 7,4′-Dihydroxyflavone; 6,4′-Dhydroxyflavone; 5,4′-Dihydroxyflavone; 3,4′-Dihydroxyflavone; 7,2′-Dihydroxyflavone; 7,3′-Dihydroxyflavone; Analog 13; Genistein; or Equol, A10, A13, A15, or a pharmaceutically acceptable salt thereof.

3. The compound, or pharmaceutically acceptable salt thereof, of claim 1, wherein the ERβ-selective compound is not an agonist, an antagonist or a mixed agonist/antagonist of estrogen receptor alpha.

4. An estrogenic combination comprising two or more ERβ-selective compounds, or pharmaceutically acceptable salts thereof, of any of claim 1.

5. A pharmaceutical composition comprising (1) one or more pharmaceutically acceptable ingredients other than an ERβ selective compound and (2) a compound of any of claim 1.

6. The pharmaceutical composition of claim 5, wherein at least one pharmaceutically acceptable ingredient other than an ERβ-selective compound is estradiol or other ERα-modulating compound.

7. The pharmaceutical composition of claim 5, wherein the at least one pharmaceutically acceptable ingredient other than an ERβ-selective compound does not occur naturally with the ERβ-selective estrogenic compound in nature.

8. The pharmaceutical composition of claim 7, further comprising a member of the group consisting of estradiol (E2), one or more estrogen receptor agonists, one or more estrogen receptor antagonists, one or more mixed estrogen receptor agonist/antagonists, one or more selective estrogen receptor modulators (SERMs), one or more progestogens, one or more glucocorticoids, and one or more androgens.

9. (canceled)

10. (canceled)

11. (canceled)

12. (canceled)

13. (canceled)

14. (canceled)

15. (canceled)

16. (canceled)

17. (canceled)

18. A method of treating a patient in need of such treatment, wherein the patient in need of treatment has a condition or disease state, the method comprising administering to the patient an effective amount of a compound of claim 3.

19. The method of claim 18, wherein the condition or disease state is a development condition, a menstruation condition, a fertility condition, a gynecological disease, an autoimmune disorder, a menopause disorder, an aging disorder, or cancer.

20. The method of claim 18, wherein the condition or disease state is Turner Syndrome, Kallman Syndrome, congenital primary amenorrhea, a childhood neuropsychiatric disorder, dysmenorrhea, amenorrhea, menorrhagia, estrogen-induced deep vein thrombosis, pulmonary embolism, conception, fetal implantation, spontaneous abortion, preterm labor, endometriosis, polycystic ovarian syndrome, rheumatoid arthritis, scleroderma, Sjogren's syndrome, Hashimoto's thyroiditis, multiple sclerosis, irritable bowel syndrome, ulcerative colitis, Crone's disease, vasomotor symptoms of menopause and perimenopause, insomnia, nighttime wakening, mood swings, vulvovaginal atrophy, vaginal dryness, dyspareunia, menopausal weight gain and obesity, osteoporosis, type-2 diabetes, estrogen-induced deep vein thrombosis and pulmonary embolism, Alzheimer's disease, early dementia, breast cancer, uterine cancer, ovarian cancer, prostate cancer, non-small cell lung cancer, and colon cancer.

21. The compound of claim 1, or a pharmaceutically acceptable salt thereof, wherein the ERβ-selective estrogenic compound is a member of the group consisting of: Liquiritigenin; Apigenin; Luteolin; Galangin; Naringenin; Calycosin; 6-methoxyluteolin; Nyasol; Wogonin; Broussonin A; 7,4′-Dihydroxyflavone; 6,4′-Dhydroxyflavone; 5,4′-Dihydroxyflavone; 3,4′-Dihydroxyflavone; 7,2′-Dihydroxyflavone; 7,3′-Dihydroxyflavone; Genistein; or Equol, or a pharmaceutically acceptable salt thereof.

22. The compound of claim 1, or a pharmaceutically acceptable salt thereof, wherein the ERβ-selective estrogenic compound is a member of the group consisting of: A10, A13, A15, or a pharmaceutically acceptable salt thereof,

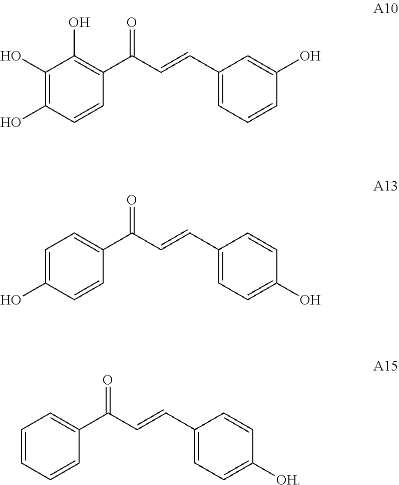 [SMILES: O=C(/C=C/c1cccc(O)c1)c1ccc(O)c(O)c1O ; O=C(/C=C/c1ccc(O)cc1)c1ccc(O)cc1 ; O=C(/C=C/c1ccc(O)cc1)c1ccccc1]

23. A pharmaceutical composition comprising a compound of claim 22, or a pharmaceutically acceptable salt thereof, and a pharmaceutically acceptable excipient.

24. The compound of claim 22, or a pharmaceutically acceptable salt thereof, wherein the ERβ-selective estrogenic compound is A10, or a pharmaceutically acceptable salt thereof.

25. A pharmaceutical composition comprising a compound of claim 24, or a pharmaceutically acceptable salt thereof, and a pharmaceutically acceptable excipient.

26. The compound of claim 22, or a pharmaceutically acceptable salt thereof, wherein the ERβ-selective estrogenic compound is A13, or a pharmaceutically acceptable salt thereof.

27. A pharmaceutical composition comprising a compound of claim 26, or a pharmaceutically acceptable salt thereof, and a pharmaceutically acceptable excipient.

28. The compound of claim 22, or a pharmaceutically acceptable salt thereof, wherein the ERβ-selective estrogenic compound is A15.

29. A pharmaceutical composition comprising a compound of claim 28, or a pharmaceutically acceptable salt thereof, and a pharmaceutically acceptable excipient.
